(19) United States
(12) Patent Application Publication
(10) Pub. No.: US 2011/0098646 A1
(43) Pub. Date: Apr. 28, 2011

(54) UNIVERSALLY APPLICABLE, OPTIMIZED PERFUSION SYSTEM

(76) Inventors: Oliver Moellenberg, Holzkirchen (DE); Frank Stickel, Stuttgart (DE); Rudolf Kober, Baden-Baden (DE); Ulrich Haag, Bisingen (DE); Mathias Nakel, Burladingen (DE)

(21) Appl. No.: 12/993,621

(22) PCT Filed: May 22, 2009
(86) PCT No.: PCT/EP09/03625

§ 371 Date: Dec. 23, 2010

(30) Foreign Application Priority Data
May 23, 2008 (DE) 102008024835.5

(51) Int. Cl. A61M 5/142

(52) U.S. Cl. 604/151

(57) ABSTRACT

The invention relates to a method of establishing and optimizing, in a perfusion system with two independently regulatable pumps, at least two independently regulatable circulations which are interconnected via at least two junction points and contain substantially the same fluid, in particular blood, blood plasma or electrolyte solutions. The perfusion system may comprise any standard devices such as those for pumping, transfer, flow or pressure regulation, filtration, bubble elimination, substance and energy exchange, or measurement of physical/chemical parameters of the fluid.

The present invention relates to a method of establishing and optimizing, in a perfusion system with two independently regulatable pumps, at least two independently regulatable circulations which are interconnected via at least two junction points chosen at will and contain substantially the same fluid, in particular blood, blood plasma or electrolyte solutions. These two circulations are: the patient circulation with the patient blood flow i.e. the flow of blood drawn from the patient and returned to the patient; and the treatment circulation with the treatment blood flow which comprises the blood flow through the blood treatment apparatus. The perfusion system may comprise any standard devices such as those for pumping, transfer, flow or pressure regulation, filtration, bubble elimination, substance and energy exchange, or measurement of physical/chemical parameters of the fluid.

The present invention moreover relates to apparatuses comprising at least one perfusion system according to the invention.

### BACKGROUND

Known perfusion systems utilize a common pump for the transfer and treatment of a given type of fluid, and in certain situations this creates disadvantages in relation to an optimum setup. For example, the blood flow through an oxygenator (a device for raising the blood's oxygen level) should never be allowed to fall below a specified minimum; this is in order to minimize clotting (coagulation). If the flow of blood to the patient has to be stopped in state-of-the-art systems (see below), flow through the oxygenator also stops in consequence. By splitting perfusion systems into a patient blood flow and a treatment blood flow each with its own independent drive unit (pump), optimal conditions can be achieved in these situations, as shown by way of example in the embodiments of the invention.

### STATE OF THE ART

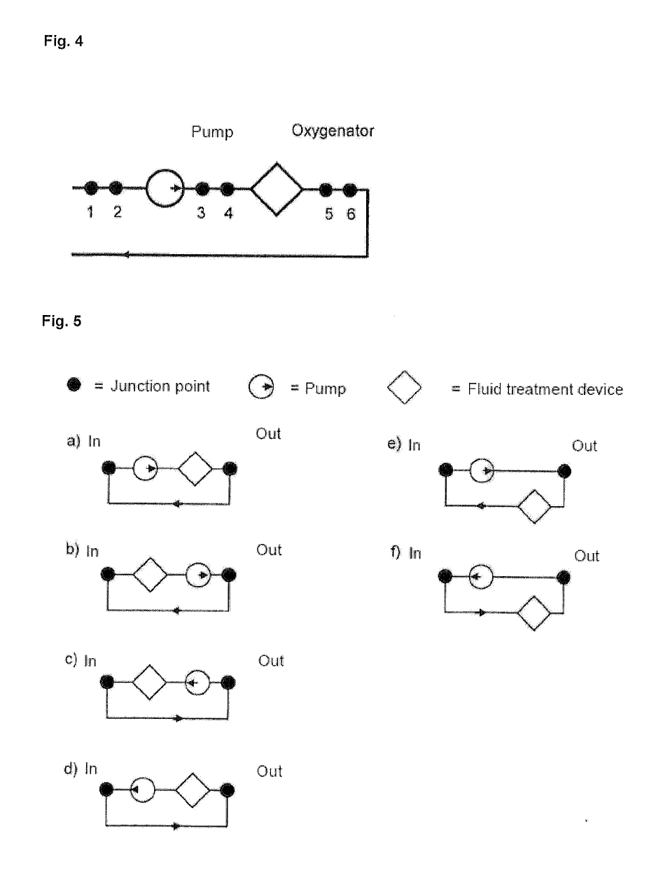

Perfusion systems, including those with several branches, have been known for quite a long time in the state of the art, e.g. as in H. Frerichs: Extracorporeal Circulation in Theory and Practice, published by Rudolf J. Tschaut, Pabst Science Publishers, 2005, page 289, FIGS. 3 and 4.

The two standard systems normally used in cardiac operations are:

1. The closed system

2. The open system.

In a closed system, the venous blood withdrawn on the patient side is fed to the extracorporeal treatment apparatus via a soft reservoir bag. Thus the extracorporeal system can be sealed off from the atmosphere. in the open system, on the other hand, a hard-shell reservoir is used, which means that there must always be an open communication between the interior of the reservoir and the atmosphere for pressure equalization purposes.

Beyond the systems which have just been mentioned, there are possible arrangements which additionally comprise an arteriovenous shunt that can be regulated e.g. by valves or clamps. This does allow a partial decoupling of the patient blood flow from the treatment blood flow; but the flow conditions which become established can be influenced only within the bounds of the prevailing a/v pressure conditions and the flow resistance. Patient blood flow and treatment blood flow cannot be stably regulated in this way; and depending on the prevailing pressure conditions, the patient flow may come to a standstill or even change direction.

Where blood-conveying products are connected in series, the blood flow through all these products is equally high, and corresponds to the total blood flow (patient blood flow). Therefore only one blood flow monitor, and only one regulatable drive pump, are needed. Nevertheless this means that compromises always have to be made in terms of the performance of the components of the apparatus, and of blood damage; and in the event of clotting of one component the entire blood flow will come to a stop. The sole alternative that exists in the state of the art is a partial or complete bypassing of individual equipment components that do not need this total blood flow. But a higher flow than the total flow in one or more apparatus or equipment components is not an option.

In known perfusion technology, besides the cardiopulmonary bypass, two further coupled but independently regulatable circulations exist in the state of the art, namely the cardioplegic circulation and the vent circulation. Other additional circulations may be established in individual cases, as has been described by D. Schwartz: Tube systems—the industry view, Extracorporeal Circulation in Theory and Practice, published by Rudolf J. Tschaut, Pabst Science Publishers, 2005, page 294, FIG. 4.

However, these circulations each driven by its own pump are only connected to the cardiopulmonary circulation at a single junction; the second contact with the patient circulation is made via the catheter/cannula and the vent, respectively.

All these known perfusion systems have the disadvantage that the independent circulations all need separate access points for the inflow or outflow of e.g. patient blood, so that additional cannulae/catheters and tube systems may be required. This implies a higher priming volume and involves greater injury to the patient due to the additional wound surfaces. Moreover, the independent circulations must all be monitored separately in this case.

Also known from the state of the art are, for example, methods and apparatuses for combined haemodialysis and CO₂ elimination that include one or more pumps.

EP 1522323 A1 discloses an apparatus consisting of an oxygenator with downstream dialyzer incorporated in a single unit. The use of such an apparatus in a system for combined haemodialysis and CO₂ elimination is disclosed in EP 1522323 A1, EP 1524000 A8 and EP 1698362, and also in WO 2005/075007 A1. WO 2005/075007 A1 discloses a second circulation which is regulatable by a regulatable pump and which leads ultrafiltrate from the ultrafiltrate outlet back to the blood inlet of the oxygenator via two junction points, thus affording the possibility of diluting the blood passing through the oxygenator. Nevertheless this circulation is not independent of the patient blood flow, as the ultrafiltrate represents at most only a fraction of the blood passing through; nor are the fluid in the patient circulation and that in the treatment circulation substantially the same (blood and ultrafiltrate respectively).

U.S. Pat. No. 5,411,706 (Hubbard et al.) describes a system consisting of a pump and an oxygenator in which an internal circulation can be set up via the outlet of the oxygenator and a recirculation line to the inlet of the pump, and reduced by more or less obstruent clamping of the recirculation line. The object is to improve oxygenator performance by this partial recirculation and by the multiple passes through the oxygenator which result. However, the patient blood flow cannot be regulated independently of the treatment blood flow. When the recirculation line is completely closed, patient blood flow reaches its maximum and is then equal to the treatment blood flow. In all other opening states of the recirculation line, patient blood flow is lower than the treatment blood flow. An added drawback of this system is that the ratio of patient blood flow to treatment flow that is set by clamping does not remain constant but may vary depending on the flow resistances prevailing in the venous or arterial line. In the extreme case, e.g. when the recirculation line is relatively wide-open and the patient's vascular resistance is rising, the patient blood flow may even come to a complete stop.

U.S. Pat. No. 3,890,969 (Fischel) describes a perfusion system which receives venous blood in a reservoir bag from which it is directed by means of an oxygenation pump through a membrane oxygenator and heat exchanger into a second reservoir bag. From the second reservoir bag, the blood is then directed back to the patient arterially via the main pump. There is a recirculation line from the second reservoir to the first reservoir to serve as an overflow for excess blood and thus prevent the second reservoir from bursting. The oxygenation pump is governed by the main pump via a control mechanism consisting of level sensor, amplifier and servo motor. The whole arrangement is intended to prevent problems due to insufficient quantities of blood. Provision must therefore be made, first of all, to match the arterial flow to the passive venous return flow so that the two flows are the same. In addition, however, the oxygenation pump must always deliver slightly more blood than the main pump, in order that the second reservoir can never be pumped empty, with the attendant risk of cavitation, formation of bubbles, and haemolysis of the blood. The two pumps, and hence the two circulations, are interdependent, and are connected in series, each with interposition of a reservoir bag; and the system has a high priming volume owing to the additional reservoirs and tube lines. Because the reservoir bags are collapsible, the system must be topped up with fluid if there is a reduction in the quantity of blood.

The problem of the invention, therefore, is to overcome the drawbacks seen in the state of the art and to provide a method and an apparatus for a universal perfusion system allowing patient fluid flow and treatment fluid flow to be regulated independently.

This problem is solved completely by the present invention and the patent claims in which the invention is expressed.

The perfusion system according to the invention makes use of two independently regulatable pumps. The first pump maintains the patient blood flow i.e. the desired venous and arterial flow. The second pump is able to pass fluid selectively through parts of the perfusion system at a rate of flow (treatment blood flow) and pressure that are optimized for the particular application. By uncoupling components from their normal series connections and reinserting the inlets and outlets at whatever junction points are best, and by controlling flow through this separate circulation by means of a regulatable pump, optimized conditions can be set up for every requirement regarding priming volume, product performance, treatment blood flow, patient blood flow, and blood damage. No additional cannulae/catheters, and hence no additional surgical interventions, are required, and only the safety-relevant patient blood flow need be monitored, as before. In contrast to the state of the art, the treatment blood flow (and associated parameters) can be set at any desired level, and can therefore be higher than the patient blood flow. Because the perfusion system according to the invention does not have to include any expandable or collapsible fluid treatment devices and the venous flow does not have to ensue on a purely passive basis, both flows are of equal magnitude, even in the absence of any equalizing control mechanism, by virtue of the incompressibility of liquids and the sealed nature of the system. The specified parameters can be reliably maintained by the independent regulation of the pumps.

With this system, the first pump, which maintains the patient blood flow and is known from the state of the art, need not be included from the outset in the perfusion system according to the invention. The perfusion system according to the invention may also be assembled by combining a conventional perfusion system with a pump for the patient circulation with the disclosed system with an independently regulatable pump for the treatment circulation.

This also includes applications using the patient's heart as a pump for the patient circulation that can be regulated independently of the treatment circulation.

Since the pumping action of the patient's heart (or the A-V pressure difference) can be influenced by medication and is therefore regulatable, the patient's heart can also function as an independently regulatable pump. Nevertheless this independence can be exploited to the full only if life-sustaining functions such as gas exchange and patient circulation can be partially or wholly taken over by connected fluid treatment apparatuses and at least one pump. Only under those circumstances can the patient's heart be adjusted downwards over an extended period, e.g. to a pumping output of nil, without causing the death of the patient.

The invention also embraces standard extracorporeal blood treatment methods such as dialysis that need an integrated blood pump in order to function. The perfusion system according to the invention with all its attendant advantages can also be adopted on such known systems by coupling an independently regulatable pump and further blood treatment devices, in accordance with the invention.

An added benefit for the patient, where further blood treatment devices with an independently regulatable pump are coupled in accordance with the invention to a blood treatment system which is already in use, is that no additional cannulae/catheters are needed.

Moreover, the possibility of combining two pumps of different types, e.g. an occlusive roller pump and a non-occlusive centrifugal pump, means that their different characteristic curves and other features such as e.g. maximum pressure generated, rate of rotation, size, electrical characteristics, suitability for long-term use, etc., can be used to good advantage, and their disadvantages (in the light of specific requirements) can be avoided. The characteristic profile resulting from such an advantageous combination of dissimilar pumps is superior to that of known single pumps and offers new treatment possibilities, whilst being adaptable to suit the specific requirement.

### DETAILED DESCRIPTION OF THE INVENTION

In the interest of simplicity, the necessary cannulae and/or catheters have been omitted from all the following descriptions and diagrams.

FIG. 1 shows the layout of a minimized closed system connected to a patient: the simplest conceivable pump-driven perfusion system. The layout of the minimized extracorporeal bypass connected to the patient is shown in highly simplified form. In FIG. 1 an oxygenator is shown as the fluid treatment device, but other fluid treatment devices such as dialyzers for example could also be included in this arrangement. Placing the fluid treatment device downstream of the pump as shown in the figure is in keeping with normal practice, but does not restrict the following disclosure of the invention to this particular case. In what would be by far the simplest case (which does not normally occur in practice), the pump could simply pump fluid back to the patient through the tube system (to support the heart's pumping function), without any other fluid treatment devices. Even then, however, it will be expedient in most cases to incorporate in the pump circuit e.g. an oxygenator to support the pulmonary function or a filter to prevent embolisms. The venous and arterial lines shown in the figure are tube connections from the patient to the pump and from the oxygenator to the patient. Another possibility is to use the pumping function of the patient's heart, or the arteriovenous (A-V) pressure difference, to drive the extracorporeal circulation. In this case, only fluid treatment devices are needed in the extracorporeal circulation; the patient blood flow is sustained by the A-V pressure difference.

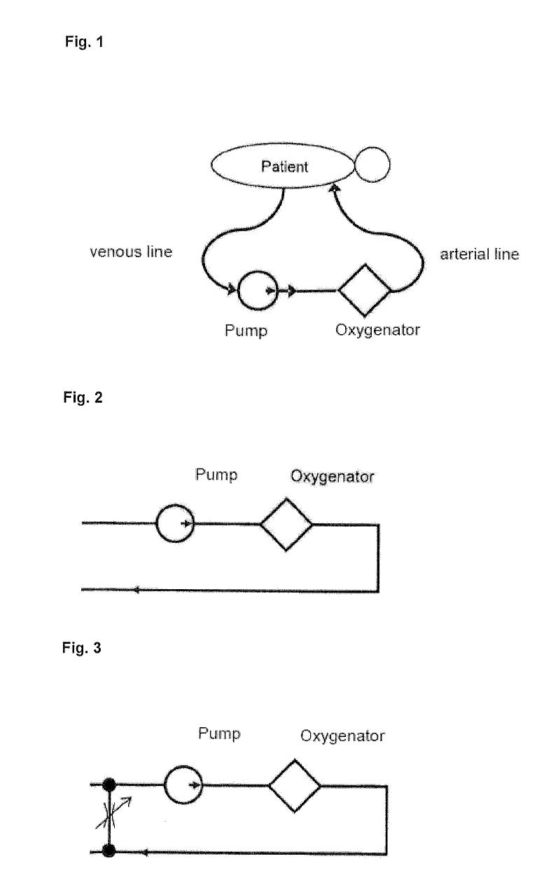

FIG. 2 shows the schematic layout of a known minimized perfusion system. In accordance with the invention, possible junction points for inlets and outlets are as indicated in FIG. 4. The arrangement has always been viewed in the direction of patient flow. Obviously, any possible inlet can be combined with any possible outlet.

FIG. 2 shows a standard circulation in which the patient blood flow is equal to the treatment blood flow. The oxygenator shown in the figure represents any desired fluid treatment devices.

FIG. 3 shows a standard circulation with provision for reducing the patient blood flow by means of a regulatable shunt. Here the patient blood flow will always be less than or equal to the treatment blood flow.

FIG. 4 shows the standard circulations from FIG. 2 and FIG. 3 with junction points according to the invention superimposed, the junctions 1, 3 and 5 representing possible blood inlets, and the junctions 2, 4 and 6, possible blood outlets. Here the patient blood flow is equal to the treatment blood flow.

The showing of only minimized systems in FIG. 2 and FIG. 4 is merely intended to make the illustration clearer, and should not be interpreted as a restriction to minimized systems.

In all the schematically shown conventional perfusion systems, any other fluid treatment devices desired in addition to those at the positions indicated may be included at any desired position of the conventional perfusion system.

Developing the known perfusion system by coupling it with a second perfusion system consisting of at least one independently regulatable pump and at least one fluid treatment device, via at least two junction points chosen at will, yields a perfusion system according to the invention with 6 possible variants in terms of the two junction points adopted for inlet and outlet (always viewing in the direction of patient flow):
Variant 1 (pump and fluid treatment device located between junctions 1 and 2)
Variant 2 (pump and fluid treatment device located between junctions 1 and 4)
Variant 3 (pump and fluid treatment device located between junctions 1 and 6)
Variant 4 (pump and fluid treatment device located between junctions 3 and 4)
Variant 5 (pump and fluid treatment device located between junctions 3 and 6)
Variant 6 (pump and fluid treatment device located between junctions 5 and 6)

Where the known perfusion system is to consist of just a pump integrated into the tube system as discussed in the description of minimized systems, the range of junction points is of course reduced to 4, resulting in only 3 different variants in this case.

Likewise, in applications using the patient's heart as the independently regulatable pump for the patient circulation, there are only 4 available junction points in the layout, of which two are located ahead of the fluid treatment device and two behind it, again resulting in only 3 different variants.

Once the possible junction points have been determined, the arrangements of pump and fluid treatment device that are possible in accordance with the invention can be considered. FIG. 5 shows the 6 possible different arrangements a) to f) of pump and fluid treatment device and the direction of flow in each arrangement, always considered in the direction of flow (in→out).

Here again only the minimum configuration (independently regulatable pump and one fluid treatment device) has been drawn, for the sake of clearer illustration.

According to the invention, however, any number of additional fluid treatment devices can be included at any desired positions in these arrangements. In the arrangements a) to f), the junction point shown on the left of the figure is always selected as the blood inlet, and the junction point shown on the right, as the blood outlet. The treatment blood flow may be selected independently of the patient blood flow in all illustrated arrangements a) to f). The junctions of FIG. 5 correspond to the junctions of FIG. 4.

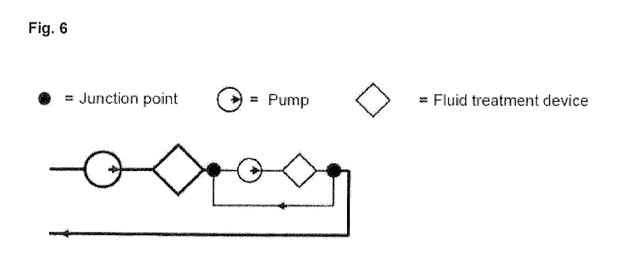

FIG. 6 shows an embodiment obtained for example by coupling the arrangement a) according to the invention to the junctions 5 and 6. Obviously, all arrangements according to the invention are possible, and FIG. 6 represents only one of the preferred embodiments. The treatment blood flow can be selected independently of the patient blood flow.

Other possible embodiments can be derived by combining the arrangements shown schematically in FIG. 4 and FIG. 5.

(A total of 6 junction-point variants×6 arrangements=36 possible alternatives.)

The functional configuration of the perfusion system can therefore be optimized for any given requirement by selecting at will the most favourable junction points and the most favourable arrangement of fluid treatment devices; and the performance parameters of the fluid treatment devices, such as e.g. the heat or substance transfer, or conservation of blood characteristics, can be optimized through the independent fluid flows.

A whole series of benefits accrue for example in the following situations, which are in no way intended to limit the applicability of the method to these cases:

Any perfusion system with a product integrated in accordance with the invention:
Oxygenator: E.g. the patient blood flow can be reduced to zero at any time, such as when an operation on the heart has been completed and the cardiac function is being checked while the oxygenator is recirculated with e.g. 2 l/min. This prevents blood from stagnating in the oxygenator, and also prevents clotting in the oxygenator if weaning from the HLM is difficult and there is a prolonged cessation of patient flow; the system is thus available at any time for an emergency situation such as a suddenly-occurring cardiac hypofunction.
Oxygenator: E.g. the treatment blood flow can be reduced to zero at any time, such as when an oxygenator needs to be replaced. The patient blood flow can then be kept going so that there is no need for a stoppage of circulation while the oxygenator is being clamped-off and replaced.
Oxygenator: It is above all in ECMO applications that the method according to the invention offers advantages. For example, every intermediate proportional condition can be regulated, from full replacement of the patient's cardiac and pulmonary functions, characterized by a high patient blood flow and a high blood flow through the oxygenator, to a complete weaning of the patient from the machine, characterized by a very low patient blood flow and a function-sustaining blood flow through the oxygenator. This means that a cardiopulmonary recovery can be effected with minimal risk to the patient and can be kept going for a longer time, and after recovery the patient can be weaned off without risk. In case of heart/lung failure, the full cardiopulmonary function can be taken over at any time.
Oxygenator: It may be beneficial to make the blood flow through the oxygenator higher than the patient blood flow. This will create stable and functionally optimal flow conditions in the oxygenator, with the blood making more than one pass through the oxygenator or with a wholly internal recirculation taking place. For example, multiple passes through the oxygenator can be particularly effective in eliminating microbubbles, or in shifting the gas exchange (if required) closer to the equilibrium setting.
Oxygenator: The perfusion systems according to the invention can be realized to extremely good advantage through the use of integrated products (bubble trap, centrifugal pump, oxygenator, heat exchanger, filter), as this can bring about a marked improvement in e.g. priming volume, heat loss and blood damage in comparison with configurations of products performing discrete functions.
Arterial filter, bubble trap: It may be beneficial to make the blood flow through the arterial filter or through a bubble trap higher than the patient blood flow. This will create functionally optimal flow conditions in the product, with the blood possibly making more than one pass through the product, or with a wholly internal recirculation possibly taking place. For example, multiple passes through the arterial filter or bubble trap can be particularly effective in eliminating microbubbles.
Blood concentrator, dialyzer: Blood flow through a blood concentrator or dialyzer can be optimized for the specific application. Also, the blood pressure and hence the product output can be regulated separately for the treatment branch. This makes it possible to achieve optimal product performance accompanied by best possible conservation of blood characteristics in this treatment circulation.
Dialyzer: With equipment-monitored dialysis, machine stoppages due to defects or failures, and hence stopping of the pump and of the patient blood flow, can occur. If other fluid treatments are additionally coupled to this system, there is a risk of clotting due to stagnation. According to the invention, a treatment blood flow is maintained in the treatment circulation by the second pump so that stagnation and hence the risk of clotting cannot occur. Thus the function of the coupled fluid treatment is not adversely affected even in the event of breakdown.
Dialyzer: The patient blood flow in a dialysis system lies in the range of say 100-500 ml/min. If other fluid treatment devices that are optimized for higher flows than this are additionally coupled to this system, there is a risk of poor product performance and/or sometimes of clotting due to stagnation in marginal zones (of the fluid treatment devices). According to the invention, an independently regulatable treatment blood flow is maintained in the treatment circulation by the second independently regulatable pump so that stagnation and hence the risk of clotting cannot occur. In cases where for example the treated fluid needs to approximate as closely as possible to an equilibrium condition after its passage through the treatment device(s), fluid treatment devices with a large active surface area, which require higher treatment flows than the presented patient blood flow in order to function reliably, can be used. It thus becomes possible to achieve e.g. virtually complete elimination of CO₂ from a relatively low presented dialysis blood flow with easy handling (no additional cannulae/catheters; dialysis blood flow does not need to be modified), and hence a higher systemic CO₂ elimination effect in spite of the low patient blood flow.

This last application example differs markedly from the state of the art: Whereas U.S. Pat. No. 5,411,706 (Hubbard et al.) proposes that a given oxygenator should be utilized to maximum capacity (in order that smaller dimensions can be attained), the object here is to provide the ideal fluid treatment device for the particular application with the flow conditions which its function requires. What this implies in the context of approximation to equilibrium conditions is the use of devices with the largest possible and most effective possible exchange surfaces (that is to say e.g. those with the largest possible dimensions). The state of the art's objective e.g. in blood oxygenation is not conditions of equilibrium but maintenance of physiologically necessary gas partial pressures. A high partial pressure of oxygen in the supply gas brings about a high transference by virtue of the high gradient between the pO₂ in the gas phase and in the blood, if a physiological oxygen partial pressure is demanded. If the blood were brought into equilibrium with this supply gas, extremely high and therefore unphysiological oxygen partial pressures would be obtained.

Again, the elimination of CO₂ in blood oxygenation is meant, according to the state of the art, to result in physiological concentrations at the oxygenator outlet, not in near-total elimination. Therefore, with physiological conditions as the objective, the state-of-the-art approach is to work with a high gradient of the physical, chemical or biological parameters to be modified through treatment, and with a relatively small, and hence low-cost, exchange surface; establishment of equilibrium is not seen as the objective at all.

The phrase “substantially the same fluid” used above should be understood to mean that the same kind of fluid is present in each of the independent partial circulations even though the fluid in one partial circulation may exhibit certain differences in a physical, chemical or biological respect from that in another, due to the devices it has passed through, or to other manipulations.

For example, after passing through a haemoconcentrator or dialyzer the treated blood will have higher HCT values; but what is present in both inflow and outflow is blood. On the other hand, the permeate obtained represents a different fluid that is cell-free and virtually protein-free and therefore cannot be equated with blood. After passing through a leucocyte filter, such treated blood will have very few, if any, leucocytes. Nevertheless the blood containing more or less leucocyte should still be regarded as consistent with the original blood.

In the case of apheresis, in the state of the art the cell-free liquid (plasma) is continuously filtered off from the blood and is then led e.g. through adsorption columns designed to adsorb the toxic substances. The toxin-free plasma is then returned to the blood, which now has higher levels of cellular constituents. Here a clear distinction must be made between blood (with more or less cellular content) and plasma as being different kinds of fluids, whereas plasma with and without toxic substances in it counts as one and the same fluid.

In the branches of the blood/plasma stream, therefore, the method according to the invention is not evinced, since the blood and plasma streams do not contain substantially the same fluid. Within the blood stream, however, before and after its passage through the plasma filter, bearing in mind that blood of different HCT is substantially the same fluid, similar fluids do exist, so that the conditions for the method according to the invention do prevail. The same applies within the plasma stream where after passage e.g. through adsorbers, toxin-free plasma results. Here too the conditions for the method according to invention are fulfilled.

The at least two junction points of the at least two circulations necessary for the function of the perfusion system according to the invention represent in practice the boundaries of a mixing leg of patient circulation and treatment circulation. An (at least very slight) admixture of treated fluid in the patient circulation is necessary in order to obtain a treatment effect. With only an ideal single junction point of the treatment circulation, and therefore with no nodal surface connecting it with the patient circulation, no mixing of the fluid of the two circulations ensues. Therefore there must be at least two junction points set at least a very small distance apart in order for it to be possible to produce an effect in the patient circulation through admixture of treated fluid. Conversely, the resultant mixing of fluid from the patient and treatment circulations can therefore be interpreted as proof that these at least two junction points do exist. This is applicable where, in perfusion systems and/or apparatuses according to the invention, the location of the junction points cannot be (or cannot be precisely) determined visually or geometrically. In integrated products that contain interconnected fluid treatment devices in combination with a pump, it may be that e.g. only an inlet and an outlet are accessible and yet an independently regulatable treatment circulation according to the invention exists internally, consisting of the pump and at least one fluid treatment device. If the method's fundamental principle of two independently regulatable circulations is realized e.g. by coupling this integrated product with at least two internal junction points to an independently regulatable patient circulation including an independently regulatable pump, in this case with a series connection, then in this case too, a perfusion system according to the invention is created.

The disclosed method and fluid treatment devices have been discussed in the examples in a patient treatment (“online” fluid treatment) scenario. Both the method according to the invention and the apparatus can of course also be used to advantage in “offline” fluid treatments where there is no patient present, in which case the term “patient circulation” should be applied mutatis mutandis.

Of course, the perfusion system according to the invention may also include components for control and regulation such as sensors for parameters to be regulated and control circuits and final control elements in addition to the pumps disclosed, without departing from the ambit of the invention.

The invention relates to a method of establishing and optimizing, in a perfusion system with two independently regulatable pumps, at least two independently regulatable circulations which are interconnected via at least two junction points and contain substantially the same fluid, in particular blood, blood plasma or electrolyte solutions. The perfusion system may comprise any standard devices such as those for pumping, transfer, flow or pressure regulation, filtration, bubble elimination, substance and energy exchange, or measurement of physical/chemical parameters of the fluid.

1. Method for a universally applicable perfusion system with at least two pumps, characterized in that
the at least two pumps are regulated independently of each other and in that at least two independently regulatable blood circulations are operated which are connected via at least two common junction points and contain substantially the same fluid, preferably blood, blood plasma or electrolyte solutions.

2. Method according to claim 1, characterized in that
to a perfusion system including at least one independently regulatable pump, a second perfusion system including at least one independently regulatable pump is added so that
at least two independently regulatable blood circulations are operated which are connected via at least two common junction points and contain substantially the same fluid, preferably blood, blood plasma or electrolyte solutions.

3. Apparatus for a universally applicable perfusion system with at least two pumps for operating the method according to claim 1, characterized in that
at least two independently regulatable pumps, and at least two independently regulatable blood circulations which are connected via at least two common junction points and contain substantially the same fluid, preferably blood, blood plasma or electrolyte solutions, are provided.

4. Apparatus for a universally applicable perfusion system according to claim 3, characterized in that
the apparatus is intended for combination with another perfusion apparatus with at least one independently regulatable pump so that in the combination
at least two independently regulatable blood circulations which are connected via at least two common junction points and contain substantially the same fluid, preferably blood, blood plasma or electrolyte solutions are provided.
